(19) United States
(12) Patent Application Publication
(10) Pub. No.: US 2008/0096825 A1
(43) Pub. Date: Apr. 24, 2008

(54) Preparation and Use of Phlorizin Compositions

(76) Inventor: Joel R. L. Ehrenkranz, Salt Lake City, UT (US)

Correspondence Address: HELLER EHRMAN LLP, 275 MIDDLEFIELD ROAD, MENLO PARK, CA, 94025-3506, US

(21) Appl. No.: 11/738,331

(22) Filed: Apr. 20, 2007

Related U.S. Application Data
(60) Provisional application No. 60/794,216, filed on Apr. 21, 2006.
(60) Provisional application No. 60/808,018, filed on May 23, 2006.

(51) Int. Cl. A61K 31/70; A61P 19/10; A61P 25/00; A61P 3/04; A61P 3/10

(52) U.S. Cl. 514/25

(57) ABSTRACT

The present invention concerns the isolation of phlorizin from plants of the genus Lithocarpus, phlorizin-containing compositions and their uses.

This is a non-provisional application claiming priority under 35 U.S.C. §119(e) to U.S. Provisional Patent Application Ser. No. 60/794,216 filed Apr. 21, 2006 and U.S. Provisional Patent Application Ser. No. 60/808,018 filed on May 23, 2006, the entire disclosures of which are hereby incorporated by reference.

### FIELD OF THE INVENTION

The present invention concerns the isolation of phlorizin from plants of the genus Lithocarpus, phlorizin-containing compositions and their uses.

### BACKGROUND OF THE INVENTION

Phlorizin (glucose, 1-[2-(beta-D-glucopyranosyloxy)-4,6-dihydroxyphenyl]-3-(4-hydroxyphenyl)-1-propanone) is a member of the chalcone class of organic compounds. It consists of a glucose moiety and two aromatic rings joined by an alkyl spacer. Phlorizin is a natural product, which has been found in and isolated from various fruit trees, including the root bark, shoots, leaves and immature and mature fruit of apple trees, and from all parts of strawberry plants. Phlorizin has been implicated in glucose metabolism, described to enhance memory and facilitate learning, and be useful in the treatment of cancer and sickle cell anemia. It has been suggested that phlorizin can be used in the treatment of type 2 diabetes, as a weight loss agent for obesity, and in the acute management of hyperglycemia. See, e.g. U.S. Pat. Nos. 6,448,232; 4,665,058; 4,684,628; 4,760,135; and 4,840,949, co-pending application Ser. No. 11/336,629, filed on Jan. 20, 2006, and Ehrenkranz et al., Diabetes Metab Res Rev 21:31-38 (2005), the entire contents of which are hereby expressly incorporated by reference.

### SUMMARY OF THE INVENTION

In one aspect, the invention concerns a method for the production of a phlorizin composition, comprising extracting phlorizin from a plant of the Lithocarpus genus, wherein phlorizin is present in the composition in the absence of trilobtain.

In one embodiment, the plant is a Lithocarpus litseiffolius, Lithocarpus pachyphyllus, Lithocarpus hancei, or Lithocarpus variolosus species.

In another embodiment, the plant is not from a Lithocarpus litseiffolius, Lithocarpus pachyphyllus species.

In further embodiments, the extraction may directly result in a phlorizin composition not containing trilobtain, or trilobtain can be additionally removed by appropriate purification.

Phlorizin can be extracted from any aerial or subsurface part of a plant, but preferably is isolated from fresh leaves, preferably collected early in the growth cycle.

Extraction may be performed with water and/or using an organic solvent, such as alcohol or an alcoholic mixture, where the alcohol preferably is ethanol or methanol.

In a particular embodiment, extraction includes a first and a second extraction step, where the first extraction step serves to extract phlorizin into an aqueous phase, and the second extraction step serves to remove polar contaminants and other impurities. Optionally, the first extraction step is preceded by exposure of the plant material to one or more enzymes in order to release phlorizin from sequestration in organelles, from where it would be difficult to extract.

The phlorizin compositions obtained may contain phlorizin in a concentration of 0.5% to about 99.5%, or about 5% to about 95%, or about 10% to about 90%, or about 20% to about 80%, or about 30% to about 70%, or about 40% to about 60%, or about 40%, or about 50%.

The above method may additionally comprise the step of converting the composition obtained by extraction into a dietary supplement, nutriceutical or pharmaceutical product, which can be in a form suitable for any route of administration, including, without limitation, oral, topical, parental administration, and administration by inhalation.

In another aspect, the invention concerns a composition comprising a phlorizin extract from a plant of the Lithocarpus genus in the absence of trilobtain, which may optionally also contain phlorizin obtained from an apple tree.

In yet another aspect the invention concerns a composition comprising a first phlorizin extract from a plant of the Lithocarpus genus in admixture with a second phlorizin extract from an apple tree, a strawberry plant, or a plant of the genus Pieris, such as Pieris japonica. Such compositions may contain one or more of phloretin, quercetin and cathecins, in addition to phlorizin.

All compositions may be in unit dosage forms, suitable for the intended administration route.

In a further aspect, the invention concerns kits comprising the compositions herein in a container and instructions for their use.

In a still further aspect, the invention concerns a method for modifying blood glucose and/or insulin levels in a mammalian subject comprising administering to the subject an effective amount of a phlorizin composition according to the invention.

In another aspect, the invention concerns a method for the prevention or treatment of hyperglycemia in a mammalian subject comprising administering to the subject an effective amount of a phlorizin composition according to the invention.

The invention further concerns a method for the prevention or treatment of type II diabetes in a mammalian subject comprising administering to the subject an effective amount of a phlorizin composition according to the invention.

The invention also concerns a method for controlling weight in a mammalian subject comprising administering to the subject an effective amount of a phlorizin composition according to the invention.

The invention further concerns a method for increasing memory or facilitating learning in a mammalian subject comprising administering to the subject an effective amount of a phlorizin composition according to the invention.

The invention additionally concerns a method for the prevention or treatment of a parasitic disease in a mammalian subject comprising administering to the subject an effective amount of a phlorizin composition according to the invention.

In a further aspect, the invention concerns a method for the prevention or treatment of bone loss or osteoporosis in a mammalian subject comprising administering to the subject an effective amount of a phlorizin composition according to the invention.

In a still further aspect, the invention concerns a method for retarding the aging process in a mammalian subject comprising administering to the subject an effective amount of a phlorizin composition according to the invention.

In all embodiments, the mammalian subject preferably is a human.

In a preferred embodiment, in the case of oral administration, the compositions of the present invention are administered on an empty stomach, at least about 15 minutes prior to eating, in order to facilitate absorption.

In a further aspect, the invention concerns a method for method for protecting a phlorizin composition from spontaneous isomerization into trilobtain, comprising storing the composition at a temperature below 10° C., preferably below 5° C., such as, for example, between about 0° C. and 5° C.

In a still further aspect, the invention concerns a method for facilitating the absorption of phlorizin through the gut comprising administering phlorizin to a mammalian subject on an empty stomach. The mammalian subject preferably is human, and phlorizin is preferably administered about 10 to 60 minutes prior to eating, such as, for example, about 15 to 30 minutes prior to eating.

### DETAILED DESCRIPTION OF THE INVENTION

### Definitions

The terms employed throughout this application are to be construed with the normal meaning to those of ordinary skill in the art. However, applicants desire that the following terms be construed with the particular definitions as described.

“Mammal” for purposes of treatment refers to any animal classified as a mammal, including humans, domestic and farm animals, and zoo, sports, or pet animals, such as dogs, horses, cats, sheep, pigs, cattle, etc. Preferably, the mammal is human.

The term “effective amount” is used herein to refer to an amount which is required to achieve the desired effect, such as health benefit, nutritional benefit, prevention or treatment of a target disease, disorder or condition.

### DETAILED DESCRIPTION

Lithocarpus is a genus in the beech family Fagaceae. A large number of species have been listed within this genus: Lithocarpus Blume, Lithocarpus abendanonii, Lithocarpus aculeatus, Lithocarpus acuminatus, Lithocarpus aggregatus, Lithocarpus aliaoensis, Lithocarpus amherstianus, Lithocarpus amoenus, Lithocarpus amygdalifolius, Lithocarpus andelsonii, Lithocarpus angustifolius, Lithocarpus anisobalanos, Lithocarpus annamensis, Lithocarpus annamoritus, Lithocarpus apoensis, Lithocarpus apricus, Lithocarpus arcaulus, Lithocarpus areca, Lithocarpus argentatus, Lithocarpus argyrocarpus, Lithocarpus arisanensis, Lithocarpus aspericupulus, Lithocarpus atjehensis, Lithocarpus attenuatus, Lithocarpus auriculatus, Lithocarpus bacgiangensis, Lithocarpus balansae, Lithocarpus bancanus, Lithocarpus bassacensis, Lithocarpus baviensis, Lithocarpus beccarianus, Lithocarpus bennettii, Lithocarpus bentramensis, Lithocarpus benzoin, Lithocarpus bicoloratus, Lithocarpus blaoensis, Lithocarpus blumeanus, Lithocarpus boholensis, Lithocarpus bolovenensis, Lithocarpus bonnetii, Lithocarpus borneensis, Lithocarpus brachycladus, Lithocarpus brachystachyus, Lithocarpus braianensis, Lithocarpus brassii, Lithocarpus brevucaudatus, Lithocarpus brochidodromus, Lithocarpus brunneus, Lithocarpus buddii, Lithocarpus bulatus, Lithocarpus bulusanensis, Lithocarpus burkillii, cagayanensis, Lithocarpus calathiformis, Lithocarpus calolepis, Lithocarpus calophyllus, Lithocarpus cambodiensis, Lithocarpus campylolepis, Lithocarpus cantleyanus, Lithocarpus carolinae, Lithocarpus castanopsifolius, Lithocarpus castellarnauianus, Lithocarpus cathayanus, Lithocarpus caudatifolius, Lithocarpus caudatilimbus, Lithocarpus celebicus, Lithocarpus cerebrinus, Lithocarpus cerifer, Lithocarpus cheliensis, Lithocarpus chevalieri, Lithocarpus chiaratuangensis, Lithocarpus chienchuanensis, Lithocarpus chifui, Lithocarpus chinensis, Lithocarpus chittagongus, Lithocarpus chiungchungensis, Lithocarpus chrysocomus, Lithocarpus cinereus, Lithocarpus clathratus, Lithocarpus cleistocarpus, Lithocarpus clementianus, Lithocarpus clementis, Lithocarpus coalitus, Lithocarpus coinhensis, Lithocarpus collettii, Lithocarpus comptus, Lithocarpus concentricus, Lithocarpus confertus, Lithocarpus confinis, Lithocarpus confragosus, Lithocarpus conocarpus, Lithocarpus coopertus, Lithocarpus copelandii, Lithocarpus corneri, Lithocarpus cornerus, Lithocarpus costatus, Lithocarpus cottonii, Lithocarpus craibianus, Lithocarpus crassifolius, Lithocarpus crassinervius, Lithocarpus crateriformis, Lithocarpus craterophorus, Lithocarpus cryptocarpus, Lithocarpus cucullatus, Lithocarpus cuneiformis, Lithocarpus curranii, Lithocarpus curtisii, Lithocarpus cuspidatus, Lithocarpus cyathiformis, Lithocarpus cyclophorus, Lithocarpus cyrtocarpus, Lithocarpus cyrtopodus, Lithocarpus cyrtorhynchus, Lithocarpus daletensis, Lithocarpus dalbertisii, Lithocarpus damiaoshanicus, Lithocarpus daphnoideus, Lithocarpus dasystachyus, Lithocarpus dealbatus, Lithocarpus debaryanus, Lithocarpus densiflurus, Lithocarpus dictyoneurus, Lithocarpus diepenhorstii, Lithocarpus dinhensis, Lithocarpus dodonaeifolius, Lithocarpus dolichocarpus, Lithocarpus dolichostachys, Lithocarpus ducampii, Lithocarpus dunnii, Lithocarpus echinifer, Lithocarpus echinocarpus, Lithocarpus echinocupulus, Lithocarpus echinophorus, Lithocarpus echinops, Lithocarpus echinotholus, Lithocarpus echinulatus, Lithocarpus edulis, Lithocarpus eichleri, Lithocarpus elaeagifolius, Lithocarpus elatus, Lithocarpus elegans, Lithocarpus elephantum, Lithocarpus elizabethiae, Lithocarpus ellipticus, Lithocarpus elmerrillii, Lithocarpus encleisocarpus, Lithocarpus ererniticus, Lithocarpus eriobotryoides, Lithocarpus eriolepis, Lithocarpus erythrocarpus, Lithocarpus eucalyptifolius, Lithocarpus eumorphus, Lithocarpus eqyckii, Lithocarpus eyrei, Lithocarpus felconeri, Lithocarpus fengii, Lithocarpus farinulentus, Lithocarpus fenestratus, Lithocarpus fenzelianus, Lithocarpus ferruineus, Lithocarpus finetii, Lithocarpus fissus, Lithocarpus floccosus, Lithocarpus fohaiensis, Lithocarpus fordianus, Lithocarpus formosanus, Lithocarpus gagpepaininus, Lithocarpus gaoligongensis, Lithocarpus garrettianus, Lithocarpus gelinicus, Lithocarpus gigantophyllus, Lithocarpus glaber, Lithocarpus glutinosus, Lithocarpus gougerotae, Lithocarpus gracilipes, Lithocarpus grandicupulus, Lithocarpus gracillis, Lithocarpus grandifolius, Lithocarpus guinieri, Lithocarpus gymnocarpus, Lithocarpus hainanensis, Lithocarpus haipinii, Lithocarpus hallieri, Lithocarpus harnatus, Lithocarpus hencei, Lithocarpus handelianus, Lithocarpus harlandii, Lithocarpus harmandii, Lithocarpus hatusimae, Lithocarpus havilandii, Lithocarpus heliciformis, Lithocarpus hemisphaericus, Lithocarpus hendersonianus, Lithocarpus henryi, Lithocarpus himalaicus, Lithocarpus honbaensis, Lithocarpus houanglipinensis, Lithocarpus howii, Lithocarpus hui, Lithocarpus hypoglaucus, Lithocarpus hypophaeus, Lithocarpus hypophoeniceus, Lithocarpus hypoviridis, Lithocarpus hystrix, Lithocarpus imperialis, Lithocarpus impressivenus, Lithocarpus indutus, Lithocarpus intermedius, Lithocarpus indutus, Lithocarpus inversus, Lithocarpus inwinii, Lithocarpus iteaphylloides, Lithocarpus ithypyllus, Lithocarpus jacksonianus, Lithocarpus jacobsii, Lithocarpus javensis, Lithocarpus jenkinsii, Lithocarpus jensenianus, Lithocarpus jingdongensis, Lithocarpus jordanae, Lithocarpus kalmanii, Lithocarpus kamengii, Lithocarpus kawakamii, Lithocarpus kemmeratensis, Lithocarpus kenigauensis, Lithocarpus kiangsiensis, Lithocarpus kingianus, Lithocarpus kingii, Lithocarpus kochummenii, Lithocarpus kudaihoensis, Lithocarpus konishii, Lithocarpus kontumensis, Lithocarpus koordersii, Lithocarpus korthalsii, Lithocarpus kostermansii, Lithocarpus kozlovii, Lithocarpus krempfii, Lithocarpus kuarunensis, Lithocarpus kunstleri, Lithocarpus kwangtungensis, Lithocarpus laetus, Lithocarpus lampadarius, Lithocarpus lampongus, Lithocarpus laoticus, Lithocarpus laouanensis, Lithocarpus lappaceus, Lithocarpus lauterbachii, Lithocarpus leiocarpus, Lithocarpus leiophyllus, Lithocarpus leiostachyus, Lithocarpus lemeeanus, Lithocarpus lepidocarpus, Lithocarpus leptogyne, Lithocarpus leucodermis, Lithocarpus leucostachyus, Lithocarpus levis, Lithocarpus licentii, Lithocarpus lindleyanus, Lithocarpus lipacon, Lithocarpus listeri, Lithocarpus lithocarpaeus, Lithocarpus litseifolius, Lithocarpus llanosii, Lithocarpus loheri, Lithocarpus longanides, Lithocarpus longicaudatus, Lithocarpus longinux, Lithocarpus longipedicellatus, Lithocarpus longipes, Lithocarpus longispinus, Lithocarpus luchinensis, Lithocarpus lucidus, Lithocarpus lutchuensis, Lithocarpus luteus, Lithocarpus luzoniensis, Lithocarpus lycoperdon, Lithocarpus mabesae, Lithocarpus macilentus, Lithocarpus macphaillii, Lithocarpus magneinii, Lithocarpus magnificus, Lithocarpus maingayi, Lithocarpus mairei, Lithocarpus mariae, Lithocarpus matsudai, Lithocarpus megacarpus, Lithocarpus megalophyllus, Lithocarpus megastachyus, Lithocarpus meijeri, Lithocarpus mekongensis, Lithocarpus melanochromus, Lithocarpus melataiensis, Lithocarpus menadoensis, Lithocarpus merrittii, Lithocarpus mianningensis, Lithocarpus microbalanus, Lithocarpus microclyx, Lithocarpus microlepis, Lithocarpus microspermus, Lithocarpus milroyi, Lithocarpus milroyi, Lithocarpus minahassae, Lithocarpus moluccus, Lithocarpus monticolus, Lithocarpus mucronatus, Lithocarpus muluensis, Lithocarpus naiadarum, Lithocarpus nakaii, Lithocarpus nantoensis, Lithocarpus nariakii, Lithocarpus nebularum, Lithocarpus neorobinsonii, Lithocarpus nhatrangensis, Lithocarpus nieuwenhuisii, Lithocarpus nitidinux, Lithocarpus nitidus, Lithocarpus nodosus, Lithocarpus nymanianus, Lithocarpus oblanceolatus, Lithocarpus oblancifolius, Lithocarpus oblinguinervius, Lithocarpus obovalifolius, Lithocarpus obovatilimbus, Lithocarpus obscurus, Lithocarpus obtusifolius, Lithocarpus ochraceus, Lithocarpus oleifolius, Lithocarpus oligocarpus, Lithocarpus ollus, Lithocarpus ormalokos, Lithocarpus ombrophilus, Lithocarpus omeiensis, Lithocarpus oogyne, Lithocarpus orbicularis, Lithocarpus ovalis, Lithocarpus oreophilus, Lithocarpus pachycarpus, Lithocarpus pachylepis, Lithocarpus pachylloides, Lithocarpus pachyphyllus, Lithocarpus paihengi, Lithocarpus pakhaensis, Lithocarpus padillus, Lithocarpus palungensis, Lithocarpus papillifer, Lithocarpus papuanus, Lithocarpus parkinsonii, Lithocarpus parvulus, Lithocarpus pasania, Lithocarpus pattaniensis, Lithocarpus paviei, Lithocarpus perakensis, Lithocarpus perclusus, Lithocarpus petelotii, Lithocarpus phansipanensis, Lithocarpus philippinensis, Lithocarpus pierrei, Lithocarpus pinatubensis, Lithocarpus platycarpus, Lithocarpus platyphyllus, Lithocarpus pleiocarpus, Lithocarpus plumbeus, Lithocarpus poculiformis, Lithocarpus podocarpus, Lithocarpus polystachyus, Lithocarpus porcatus, Lithocarpus proboscideus, Lithocarpus propinguus, Lithocarpus pruinosus, Lithocarpus psammophilus, Lithocarpus pseudokunstleri, Lithocarpus pseudolaponga, Lithocarpus pseudomagneinii, Lithocarpus pseudomoluccus, Lithocarpus pseudoplatycarpus, Lithocarpus pseudoreinwartii, Lithocarpus pseudosundaicus, Lithocarpus pseudovestitus, Lithocarpus pseudoxizangensis, Lithocarpus pucher, Lithocarpus pusillus, Lithocarpus lychnostachys, Lithocarpus pyriformis, Lithocarpus qinhouicus, Lithocarpus quangnamensis, Lithocarpus quercifolius, Lithocarpus rajah, Lithocarpus randaiensis, Lithocarpus rangerianus, Lithocarpus rassa, Lithocarpus recurvatus, Lithocarpus reflexus, Lithocarpus reinwardtii, Lithocarpus revolutus, Lithocarpus rhabdostachyus, Lithocarpus thioensis, Lithocarpus rhombocarpus Lithocarpus ridleyanus, Lithocarpus rigidus, Lithocarpus rizalensis, Lithocarpus robinsonii, Lithocarpus rodgerianus, Lithocarpus rosthornii, Lithocarpus rotundatus, Lithocarpus rouletii, Lithocarpus rufescens, Lithocarpus rufuvillosus, Lithocarpus rufus, Lithocarpus ruminatus, Lithocarpus sabulicolus, Lithocarpus sandalanensis, Lithocarpus sarawakensis, Lithocarpus schlechteri, Lithocarpus scortechinii, Lithocarpus scutigera, Lithocarpus scyphiger, Lithocarpus seishoi, Lithocarpus sericobalanos, Lithocarpus shinsuiensis, Lithocarpus shunningensis, Lithocarpus siamensis, Lithocarpus sieboldii, Lithocarpus silvicolarum, Lithocarpus skanianus, Lithocarpus smitinandianus, Lithocarpus sogerensis, Lithocarpus solanicarpus, Lithocarpus soleriaus, Lithocarpus songkoensis, Lithocarpus sootepensis, Lithocarpus spaerocarpus, Lithocarpus spicatus, Lithocarpus stenopus, Lithocarpus stipitatus, Lithocarpus stone, Lithocarpus sublepidotus, Lithocarpus submonticolus, Lithocarpus subnucifer, Lithocarpus subreticulatus, Lithocarpus suffruticosus, Lithocarpus suishaensis, Lithocarpus sulitii, Lithocarpus sundaicus, Lithocarpus symingtonianus, Lithocarpus synbalanos, Lithocarpus syncarpus, Lithocarpus tabularis, Lithocarpus taitoensis, Lithocarpus talangensis, Lithocarpus tapintzensis, Lithocarpus tawaiensis, Lithocarpus tenuilinbus, Lithocarpus tenuinervis, Lithocarpus tephrocarpus, Lithocarpus tenaticupulus, Lithocarpus teysmannii, Lithocarpus thalassicus, Lithocarpus thomsonii, Lithocarpus toumorangensis, Lithocarpus touranensis, Lithocarpus trachycarpus, Lithocarpus tremulus, Lithocarpus trigueter, Lithocarpus truncatus, Lithocarpus tsangii, Lithocarpus tubulosus, Lithocarpus tubinatus, Lithocarpus uncinatus, Lithocarpus uraianus, Lithocarpus urceolaris, Lithocarpus uvatiifolius, Lithocarpus uvartifolius, Lithocarpus variolosus, Lithocarpus vestitus, Lithocarpus vidalianus, Lithocarpus dialii, Lithocarpus vinhensis, Lithocarpus vinkii, Lithocarpus viridis, Lithocarpus wallichianus, Lithocarpus wangianus, Lithocarpus wenzelii, Lithocarpus wensigianus, Lithocarpus winklerianus, Lithocarpus wodit, Lithocarpus woonyoungii, Lithocarpus wrayi, Lithocarpus xizangensis, Lithocarpus sylocarpus, Lithocarpus yersinii, Lithocarpus youngfiuensis, Lithocarpus zschokei. In addition, many of the listed species have several known variants.

All but one Lithocarpus species are native to East and South East Asia. The only exception is Lithocarpus desiflorus (tanoak), which is native to Western North America, especially Oregon and California.

Species particularly suitable for the present invention include Lithocarpus litseiffolius, Lithocarpus pachyphyllus, Lithocarpus hancei, and Lithocarpus variolosus.

Extracts from various anatomical parts, such as leaves, twigs, stem bark and roots of Lithocarpus species have been used by indigenous peoples as a medical plant and screened by scientists to identify biologically active substances. Extracts obtained from Lithocarpus pachyphyllus and Lithocarpus litseiffolius have been reported to contain phlorizin accompanied by trilobtain (Qin et al., Z. Naturforsch. 58(9-10):759-761 (2003); Yang et al., Z, Naturforsch. 59c:481-484 (2004); Nie et al, Agric. Biol. Chem. 46:1933-1934 (1982)). There have been no reports of the extraction of phlorizin from other Lithocarpus species, or the preparation of extract from Lithocarpus pachyphyllus and Lithocarpus litseiffolius in the absence of trilobtain.

The present invention provides phlorizin compositions obtained from a plant belonging to the Lithocarpus genus. Phlorizin isomerizes to trilobtain at room temperature which leads to phlorizin degradation. Accordingly, preferably, the compositions contain phlorizin in the absence of trilobtain. This result can be obtained in several ways. Thus, for example, phlorizin can be extracted from a plant belonging to the Lithocarpus genus by selective extraction, which belonging to the Lithocarpus genus, to yield a composition which is substantially free of trilobtain. Alternatively, trilobtain can be removed from the phlorizin composition following extraction by a purification method known in the art.

According to the present invention, phlorizin can be extracted from any parts of a plant belonging to the Lithocarpus genus, including, without limitation, the all above-described species, and their variants. In a particular embodiment, phlorizin is isolated from Lithocarpus litseiffolius Lithocarpus pachyphyllus, Lithocarpus hancei, or Lithocarpus variolosus, most preferably from Lithocarpus litseiffolius. The phlorizin extract may be derived from any part of the plant, including, without limitation, all aerial and subsurface parts, such as leaves, twigs, bark, roots, and fruit. Pholorizin extracts may be produced, for example, by water-based extraction of target materials by soaking parts of the plant in water. Extraction may also be effected by use of alcohols, such as ethanol or methanol, and/or other suitable solvents, as well as by supercritical CO₂ extraction.

A typical extraction method will include the following steps:

1. mechanical chipping/grinding/shredding/pulverizing of plant material;

2. extraction into an aqueous phase;

3. filtration to remove particulate matter;

4. second extraction to remove polar contaminants and other impurities;

5. chromatography;

6. purification, including decolorizing and drying.

If desired, step 1 may be followed by the optional step of exposing the plant material to one or more enzymes which may release phlorizin from sequestration in organelles that otherwise could limit extraction. Suitable enzymes include, for example, cellulose enzymes, dextranase, chitinase, lysozyme, trehelase, cermidase, fucosidase, agarase, chlorophyllase, and tannase.

The first extraction step (step 2 in the above scheme) is typically carried out with a warm or hot organic solvent or solvent mixture, or a saline solution, at atmospheric or increased pressure. The organic solvent can, for example, be an alcohol, such as methanol, ethanol, propanol, isopropanol, butanol, sec-butanol or tert-butanol; an ester, such as, for example, methyl formate, ethyl formate, propyl formate, isopropyl formate, methyl acetate, ethyl acetate, propyl acetate, isopropyl acetate, butyl proprionate, isobutyl propionate, methyl butyrate, ethyl butyrate, propyl butyrate, isopropyl butyrate, isobutyl butyrate; a ketone, such as, for example, acetone, methyl ethyl ketone, methyl propyl ketone, methyl isopropyl ketone, methyl butyl ketone, methyl isobutyl ketone, methyl tert-butyl ketone, diethyl ketone, diisopropyl ketone, methyl vinyl ketone, cyclobutanone, cyclopropanone, cyclopentanone, cyclohexanone; hydrocarbons, such as, for example, pentane, hexane, heptane, octane, nonane, decane, nonadecane, cyclohexane, xylene, toluene; ethers, such as tetrahydrofurane, 1,2-dimethoxyethane, diisopropyl ether; nitrites, such as acetonitrile; carboxylic acids, such as acetic acid or methyl acetate, or appropriate mixtures thereof. Selection of the solvent or solvent mixture and the extraction conditions, including temperature, will depend on the desired composition of the phlorizin-containing polyphenol extract, including phlorizin concentration and the nature and quantity of other polyphenols and additional non-polyphenol components present.

The extract is allowed to cool, and filtered to remove particulate matter.

Before the second extraction step (step 4 in the above scheme), the organic solvent is evaporated, to yield an oily mixture, followed by the addition of an organic solvent, which may be selected, for example, from the alcohols, ethers, esters, hydrocarbons, ketones, nitriles, carboxylic acids, listed above, or can be chloroform. The second extraction step is followed by atmospheric distillation and trituration to yield a solid or tar-like material.

In the next step, polar impurities are removed by partitioning the solid or tar-like material between non-polar organic solvent (e.g. ethyl acetate) and water, and discarding the aqueous phase.

Purification can be performed by a variety of chromatography techniques, including, for example, silica gel or SEPHADEX® liquid phase chromatography, where the mobil phase can consist of one or more of the following solvents: esthers, esters, ketones, hydrocarbons, alcohols, nitrites, carboxylic acids. For representatives of these solvent groups, see the list provided above.

If desired, the material obtained by chromatography can be further purified by methods known in the art. Further purification may, for example, include one or more of the following steps: crystallization, treating with a decolorizing agent (e.g. charcoal), and distillation to remove alcohol.

The phlorizin extracts typically will contain about 0.5% to about 99.5% phlorizin, such as about 5% to about 95% phlorizin, or about 10% to about 90% phlorizin, or about 20% to about 80% phlorizin, or about 30% to about 70% phlorizin, preferably about 40% to about 60% phlorizin, most preferably about 40% or about 50% phlorizin. In addition to phlorizin, the extracts may contain other polyphenols, in particular flavonoids, including, without limitation, chalcones, flavon, flavonol, flavanone, anthocyanines, and/or isoflavonoids, as well as phloretin, catechins, quercetin, and polymers thereof, which may or may not have biological activities on their own.

Extracts of higher purity, such as 99% or above, can be obtained by various methods, such as by increasing the selectivity of the extraction process, and optionally further purification by methods known in the art, such as chromatographic techniques. Such high purity phlorizin preparations are specifically within the scope of the present invention, and are particularly suitable for pharmaceutical uses, especially for parenteral or topical administration, which will be discussed in greater details below.

The invention further extends to phlorizin compositions, which include mixtures of phlorizin preparations, such as extracts, obtained from various sources, including mixtures of phlorizin and/or phlorizin extracts obtained from one or more of different Lithocarpus species, various parts of apple tree and strawberry plants. In a specific embodiment phlorizin or phlorizin extract obtained from a Lithocarpus species is mixed with phlorizin or extract obtained from an apple tree. In the latter case, in the mixture obtained phlorizin may be accompanied by one or more of phloretin, quercetin, and catechins.

Phlorizin extracts have been demonstrated to have beneficial effects in producing glucosuria, decreasing postprandial rise in glucose and insulin levels, elimination of reactive hypoglycemia, control of stress hyperglycemia, weight loss, and in preventing weight gain. In addition, phlorizin is believed to be effective in enhancing memory and facilitating learning, and has been suggested for use in the treatment of type 2 diabetes. Phlorizin can also be used to treat parasitic diseases, prevent or treat osteoporosis, and to retard the aging process.

The phlorizin extracts can be formulated as a dietary supplement, or nutriceutical or pharmaceutical product, depending on the intended use. Thus, without limitation, the phlorizin extracts can be incorporated into food products, formulated as food or dietary supplements, consumed as tea or other beverages, or converted into dietary, pharmaceutical or cosmetic formulations, for oral, injectable, or topical use.

In particular, if the phlorizin extract is used, directly or after further processing, as a dietary supplement or nutriceutical, it can, for example, be formulated in any unit dosage form suitable for oral or topical administration, including tablets, capsules, pills, powders, lotions, ointments, salves, gels, foams, sprays, creams, lozenges, and the like, in admixture with further ingredients commonly used in dietary supplements or nutriceuticals. In such forms, the preparation is subdivided into unit doses containing appropriate quantities of the nutriceutically active component. The unit dosage form can be a packaged preparation, such as packeted tablets, capsules, lotions, ointments and powders in vials or ampoules. Also, the unit dosage can be a drink, such as a powder-based drink, drink mix, shake or tea formulation, yoghurt, or a solid food product or chewing gum. It is noted that these forms are merely illustrative and further forms will be apparent to and can be readily identified by those skilled in the art.

All forms may be optionally supplemented with additives improving palatability, appearance and/or providing further health benefits, such as other flavonoids, dietary supplements, vitamins, and essential minerals. Thus, a dietary supplement of the present invention may, for example, be formulated with one or more of the following additional ingredients: vitamin A, vitamin A acetate, vitamin B₁, vitamin B₂, vitamin B₆, vitamin B₁₂, vitamin D, calcium, niacinamide, copper, iodine, iron, magnesium, manganese, hydrochloride and selenium. The metal ingredients can be incorporated in the form of their salts, such as, for example, sulfates, citrates, or carbonates. Other components, such as soluble fiber compounds, e.g. locust gum, guar gum, pectin, gum arabic, and/or psyllium may also be included and are well known to those skilled in the art.

When the primary goal is weight loss, the phlorizin extracts of the present invention can be generally used in any of the above-described forms, including all forms suitable for transdermal (topical) administration, such as skin patches. The patches are applied to a skin, and allow the active ingredient(s) to be transdermally absorbed into the body. The patches have various benefits over other forms of topical administration, such as ointments, including accuracy of the applied dose and simplicity of administration. In addition, the patches allow the drug to be continuously absorbed, thereby showing a prolonged action. This prolonged action is particularly beneficial when one of the goals is weight reduction which results from phlorizin's effects on glucose metabolism. Substrates suitable for making skin patches are well known in the art, and include polyester, polyvinyl chloride, lint nylon, an unwoven fabric or a composite material. If necessary, a liner of a suitable material (such as a polypropylene film, polyethylene film, polyurethane film and the like) can be attached to the surface of the drug reservoir layer in order to prevent evaporation of water, and to protect the layer. The thickness of the substrate is not particularly limited and can be appropriately chosen depending on the applications.

Pharmaceutical compositions for therapeutic use, specifically including, without limitation, any and all therapeutic uses listed above, may take the form of any of the formulations suitable for oral administration discussed above or otherwise known in the art.

Pharmaceutical compositions suitable for injectable use include sterile aqueous solutions (where water soluble) or dispersions and sterile powders for the extemporaneous preparation of sterile injectable solutions or dispersion. Aqueous injection suspensions may comprise substances which increase the viscosity of the suspension, such as sodium carboxymethyl cellulose, sorbitol, or dextran. Optionally, the suspension may also comprise suitable stabilizers or agents which increase the solubility of the compounds to allow for the preparation of highly concentrated solutions. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes.

For intravenous administration, suitable carriers include, for example, physiological saline, bacteriostatic water, Cremophor EL™ (BASF, Parsippany, N.J.) or phosphate buffered saline (PBS).

In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium comprising, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. The presence of buffers and/or stabilizing agents may also be beneficial in order to prevent the degradation of phlorizin, especially when trilobtain is present. Also, in order to prevent degradation, the phlorizin formulations herein are preferably stored at cool temperatures (e.g. around 5° C.) in order to prevent or, at least, slow down degradation, especially in the absence of an antibacterial or other stabilizing agent or buffer.

The compositions of the present invention may also be administered intranasally or by inhalation. For administration intranasally or by inhalation, the phlorizin extracts of the present invention are conveniently delivered in the form of an aerosol spray from pressurized packs or a nebulizer, using a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated comprising a powder mix of the active ingredient(s) and a suitable powder base such as lactose or starch.

In addition, skin patches, as described above, may also be suitable for pharmaceutical use.

In order to improve bioavailability of the phlorizin extracts and/or to extend the duration of biological activity, the extracts may be administered in a form of a complex, for example with phosphatidylcoline, or delivered in the form or a solution or suspension with one or more organic solvents, such as polypropylene glycol. Of course, other additives and methods known in the art to increase bioavailability and/or prolong the duration of biological activity can also be used.

The extracts and compositions herein may additionally include, or administered in combination with, phlorizin and/or other polyphenols, especially flavonoids, obtained from other sources or prepared by other methods. In addition, phlorizin extracts and compositions may be used in combination with other supplements, or pharmaceutical compositions, effective to treat the target disease. Thus, for example, if the intended use is control of blood and/or urine glucose levels, including the prevention or treatment of glucosuria, decrease of postprandial rise in glucose and insulin levels, elimination of reactive hypoglycemia, control of stress hyperglycemia, or prevention or treatment of type II diabetes, the extracts and compositions herein may be administered in combination with insulin, insulin sensitizers, insulin secretagogues, inhibitors of hepatic gluconeogenesis, and/or alpha glucosidase inhibitors, as well as with other natural products having blood glucose lowering properties. Any reference herein to administration “in combination” with an additional substance or preparation includes administration from separate formulations, concurrently or consecutively in any order, or from the same formulation.

When the extracts and compositions herein are used to prevent or treat osteoporosis, they can be administered in combination with other medications, including herbal extracts, dietary supplements and pharmaceutical compositions, used for this purpose. Suitable pharmaceutical compositions include, without limitation, ACTONEL® (risedronate sodium), BONIVA® (ibandronate sodium), and FOSAMAX® (etidronate). Dietary supplements typically contain a form of calcium, such as calcium citrate or calcium carbonate, in combination with Vitamin D and optionally other vitamins.

The daily dosage will depend on the intended use, and the condition, age, sex, overall health of the subject to which the compositions are administered. Determination of the appropriate daily dose is well within the skill of an ordinary artisan. Typically, for all uses contemplated herein, the daily dose is expected to be between about 3 grams and about 30 grams of the phlorizin extract, however, the dosage may vary, depending on the phlorizin concentration in the abstract. Administration typically will be three-times a day, although other, e.g. twice or four-times daily, schedules might also be suitable.

Bioavailability of phlorizin may be enhanced by various methods, including, for example, a dosing schedule when phlorizin is ingested on an empty stomach, about 10-60 minutes, such as about 30 minutes, prior to eating. Another suitable dosing schedule is administration on an empty stomach, about 10-20 minutes, such as about 15 minutes, prior to eating.

While the present invention has been illustrated by reference to certain embodiments, it is not so limited. Indeed, various modifications of the invention in addition to those shown and described herein will become apparent to those skilled in the art from the foregoing description and fall within the scope of the appended claims.

What is claimed is:

1. A method for the production of a phlorizin composition, comprising extracting phlorizin from a plant of the Lithocarpus genus, wherein said phlorizin is present in said composition in the absence of trilobtain.

2. The method of claim 1 wherein said plant is selected from the group consisting of Lithocarpus litseiffolius, Lithocarpus pachyphyllus, Lithocarpus hancei, and Lithocarpus variolosus.

3. The method of claim 1 wherein said plant is other than Lithocarpus litseiffolius or Lithocarpus pachyphyllus.

4. The method of claim 1 wherein the extraction results in a composition containing phlorizin in the absence of trilobtain.

5. The method of claim 1 wherein the mixture obtained by extraction is further purified to remove trilobtain.

6. The method of claim 1 wherein the composition obtained by extraction is stored at reduced temperature, whereby spontaneous isomerization into trilobtain is inhibited.

7. The method of claim 6 wherein said temperature is about 5° C.

8. The method of claim 1 wherein said phlorizin is extracted from an aerial or subsurface part of said plant.

9. The method of claim 8 wherein phlorizin is extracted from fresh leaves of said plant.

10. The method of claim 9 wherein said fresh leaves are collected early in the leaf growth cycle.

11. The method of claim 1 wherein said extraction is performed using a first extraction step and a second extraction step.

12. The method of claim 11 wherein said first extraction step is performed with one or more hot organic solvents or a saline solution at atmospheric or increased pressure.

13. The method of claim 12 wherein said organic solvent is selected from the group consisting of alcohols, esters, ethers, ketones, hydrocarbons, nitriles, and carboxylic acids.

14. The method of claim 11 wherein said second extraction step is performed with one or more organic solvents selected from the group consisting of ethers, esters, alcohols, hydrocarbons, ketones, nitriles, carboxylic acids, and chloroform.

15. The method of claim 11 wherein the material of said plant is subjected to mechanical treatment prior to extraction.

16. The method of claim 15 wherein the mechanically treated material is treated with one or more enzymes prior to the first extraction step.

17. The method of claim 16 wherein the enzyme is selected from the group consisting of cellulose enzymes, dextranase, chitinase, lysozyme, trehalase, ceramidase, fucosidase, agarase, chlorophyllase, and tannase.

18. The method of claim 11 additionally comprising partitioning between an aqueous and a nonpolar organic phase, following said second extraction step in order to remove polar impurities.

19. The method of claim 18 further comprising a chromatographic purification step following said partitioning.

20. The method of claim 1 wherein said phlorizin composition comprises from about 0.5% to about 99.5% phlorizin.

21. The method of claim 20 wherein said phlorizin composition comprises from about 5% to about 95% phlorizin.

22. The method of claim 21 wherein said phlorizin composition comprises from about 10% to about 90% phlorizin.

23. The method of claim 22 wherein said phlorizin composition comprises from about 20% to about 80% phlorizin.

24. The method of claim 23 wherein said phlorizin composition comprises from about 30% to about 70% phlorizin.

25. The method of claim 24 wherein said phlorizin composition comprises from about 40% to about 60% of phlorizin.

26. The method of claim 25 wherein said phlorizin composition comprises about 40% phlorizin.

27. The method of claim 25 wherein said phlorizin composition comprises about 50% phlorizin.

28. The method of claim 1 further comprising the step of converting said composition into a dietary supplement, nutriceutical or pharmaceutical product.

29. The method of claim 28 wherein said dietary supplement or nutriceutical is in a form suitable for oral administration.

30. The method of claim 29 wherein said dietary supplement or nutriceutical is in the form of tablets, capsules, pills, capsules, or a powder.

31. The method of claim 28 wherein said dietary supplement or nutriceutical is in a form suitable for topical administration.

32. The method of claim 31 wherein said dietary supplement or nutriceutical is in the form of a lotion, ointment, salve, gel, foam, spray, cream, or lozenge.

33. The method of claim 28 wherein said dietary supplement or nutriceutical is in the form of a drink, powder-based drink, drink mix, shake, tea, yoghurt, solid food product, nutrition bar, or chewing gum.

34. The method of claim 28 wherein said pharmaceutical product is suitable for oral, topical, injection, or inhalation administration.

35. The method of claim 34 wherein said pharmaceutical product additionally comprises one or more pharmaceutically acceptable excipients.

36. A composition comprising a phlorizin extract from a plant of the Lithocarpus genus, wherein
a) said phlorizin is present in the absence of trilobtain; and/or
b) said composition further comprises one or more additional components selected from the group consisting of phlorizin obtained from an apple tree or a plant of the Pieris genus, phloretin, quercetin and cathecins; and/or
c) said composition comprises phlorizin in a purity of a least about 95%.

37. The composition of 36 which is a dietary supplement, nutriceutical, or a pharmaceutical composition.

38. A unit dosage form comprising the composition of claim 37.

39. The phlorizin composition of claim 37 which is in the form of a lotion, ointment, salve, gel, foam, spray, cream, or lozenge.

40. A kit comprising the composition of claim 36 in a container, and instructions for use of said composition.

41. A method for modifying blood glucose and/or insulin levels in a mammalian subject comprising administering to said subject an effective amount of a phlorizin composition according to claim 36.

42. The method of claim 41 wherein said mammalian subject is a human.

43. The method of claim 42 wherein blood glucose and/or insulin levels are decreased.

44. A method for the prevention or treatment of hyperglycemia comprising administering to a mammalian subject in need an effective amount of a phlorizin composition according to claim 36.

45. The method of claim 44 wherein said mammalian subject is a human.

46. A method for the prevention or treatment of type II diabetes comprising administering to a mammalian subject in need an effective amount of a phlorizin composition according to claim 36.

47. The method of claim 46 wherein said mammalian subject is a human patient.

48. A method for controlling weight in a mammalian subject comprising administering said subject an effective amount of a phlorizin composition according to claim 36.

49. The method of claim 48 wherein said mammalian subject is a human.

50. The method of claim 49 wherein the weight control is weight reduction.

51. The method of claim 49 wherein the weight control is the prevention or reduction of weight gain.

52. The method of claim 49 wherein the weight control is the prevention or reduction of weight fluctuation.

53. A method for increasing memory or facilitating learning comprising administering to a mammalian subject an effective amount of a phlorizin composition according to claim 36.

54. The method of claim 53 wherein said mammalian subject is a human.

55. A method for the prevention or treatment of a parasitic disease comprising administering to a mammalian subject an effective amount of a phlorizin composition according to claim 36.

56. The method of claim 55 wherein said mammalian subject is a human.

57. A method for the prevention or treatment of bone loss or osteoporosis comprising administering to a mammalian subject an effective amount of a phlorizin composition according to claim 36.

58. The method of claim 57 wherein said mammalian subject is a human.

59. A method for retarding the aging process comprising administering to a mammalian subject an effective amount of a phlorizin composition according to claim 36.

60. The method of claim 59 wherein said mammalian subject is a human.

61. The method according to claim 41, wherein said administration takes place on an empty stomach, at least about 15 minutes prior to eating.

62. The method of claim 61 wherein said administration takes place on an empty stomach at least about 30 minutes prior to eating.

63. A method for protecting a phlorizin composition from spontaneous isomerization into trilobtain, comprising storing said composition at a temperature below 10° C.

64. The method of claim 63 wherein said composition is stored at a temperature of about 5° C. or below.

65. The method of claim 64 wherein said composition is stored below 5° C.

66. The method of claim 64 wherein said composition of stored between about 0° C. and about 5° C.

67. A method for facilitating the absorption of phlorizin through the gut comprising administering phlorizin to a mammalian subject on an empty stomach.

68. The method of claim 67 wherein the mammalian subject is a human.

69. The method of claim 68 wherein said phlorizin is administered on an empty stomach about 10 to 60 minutes prior to eating.

70. The method of claim 68 wherein said phlorizin is administered on an empty stomach at least about 30 minutes prior to eating.

71. The method of claim 68 wherein said phlorizin is administered on an empty stomach at least about 15 minutes prior to eating.
